(19) United States
(12) Patent Application Publication
(10) Pub. No.: US 2005/0257996 A1
(43) Pub. Date: Nov. 24, 2005

(54) Disposable cover for stethoscope head

(76) Inventors: Basil Brown, DeSoto, TX (US); Yvonne Mosley Brown, DeSoto, TX (US)

Correspondence Address: Basil Brown, P.O. Box 3473, Cedar Hill, TX, 75106, US

(21) Appl. No.: 10/033,120

(22) Filed: Oct. 29, 2001

Related U.S. Application Data
(60) Provisional application No. 60/244,344, filed on Oct. 29, 2000.

(51) Int. Cl. A61B 7/02

(52) U.S. Cl. 181/131

(57) ABSTRACT

The disposable stethoscope cover is a medical device that is designed to prevent the spread of infection by not allowing any fluids, infectious or not, to pass through it. It is a universal precaution which institutes personal protective equipment to provide barrier protection preventing liquids from transporting through it. It is designed for a single use. It is designed to be used wherever stethoscopes are used. It is designed to protect the stethoscope from contamination and to prevent the spread of contamination via the stethoscope. According to federal mandates in OSHA states, personal protective equipment should be provided to employees that perform duties that place them at risk of occupational exposure at no cost to the employees. Also, according to the mandates, the product should be disposed of by the employer with all costs of disposal being borne by the employer. Recently, non-OSHA states have enacted legislation to address the potential spread of infection through blood and bloodborne pathogens. Pursuant to this legislation, personal protective equipment should be worn at all times whenever contamination and or infection are possible. Preventing infection will aide mankind in increasing our quality of life.

### CROSS REFERENCE TO RELATED APPLICATIONS

This Utility Patent Application cross references to Provisional Patent Application No. 60/244,344 filed on Oct. 29, 2000. There are no other applications cross-referenced.

### BACKGROUND OF THE INVENTION

1. Field of the Invention

This invention relates to a necessity to increase efforts to prevent the spread of infections, diseases, body fluids, and organisms from one patient to the next, or from a patient to a medical personnel providing care to the patient, due to the use of an unsterile stethoscope being used from one patient to the next over and over again. A stethoscope cover would act as a shield that would lie between the body of a patient and the face of the medical equipment.

2. Description of the Related Art

Medical personnel do not have time to sterilize their stethoscope after each use. Thus, a disposable cover that prevents the spread of infection, diseases, body fluids, and organisms from one patient to the next is imperative. The stethoscope cover acts as a shield that lies between the body of the patient and the face of the stethoscope that actually makes contact with the patients skin. This shield will prevent, and possibly eliminate, the spread of infections, diseases, body fluids, and organisms from patient to patient in a medical setting or from person to person in a non-medical setting. This shield will also aide in eliminating nosocomial infections in a hospital, Doctor's office, or other medical environment.

Stethoscopes are used several times a day in a hospital or other medical environment. In most cases, a stethoscope is used on virtually every patient in a hospital one, or more, times per day. The stethoscopes are generally not sterilized after use from one patient to the next. Thus, the possibility of the spread of infections, diseases, body fluids, or organisms from one patient to the next is extremely high. Considering the fact that most patients in the hospital are already battling other illnesses and that their immune systems could already be weakened, adding a new infection due to the use of an unsterile stethoscope could prove to be fatal. Thus, the need for a disposable stethoscope head cover is increasingly becoming a necessity in both the hospital setting and in the non-hospital setting.

Typically, the head of the stethoscope is placed in direct contact with a patients skin. This is done in an effort to listen closely to a patient's heart rate and rhythms, lung clarity, abdominal sounds, and to determine blood pressure. Generally, this procedure is practiced on each patient in a hospital setting one, or more, times per day by one, or more, different hospital personnel. Medical personnel typically use their own personal stethoscope to perform these procedures day after day. Rarely, if ever, is a stethoscope sterilized on a daily basis, not to mention on a patient-to-patient basis. Thus, patients are exposed to a tremendous risk of contracting a harmful infection or disease due to direct contact with an unsterilized stethoscope head.

The Center for Disease Control reports that infections, diseases, body fluids, and organisms are being transferred from patient-to-patient at a much higher rate now than ever before due to the use of unsterile equipment being used in our hospitals and other medical settings such as Doctor's offices and clinics. Thus, it is imperative that Universal Precautions be established, and utilized, in the area pertaining to the use of stethoscopes and other medical equipment used on multiple patients each day, week, month, and year. Typical universal precautions encourage the use of protective gear such as gloves, masks, gowns, head covers, and shoe covers. These precautions are used to protect the patients and the medical personnel from spreading infections, diseases, body fluids and organisms to each other. The same type of Universal Precautions should be established, and utilized, in the area of medical equipment such as stethoscopes.

### BRIEF SUMMARY OF THE INVENTION

The disposable stethoscope cover is designed to provide a safer work environment for employees in the medical care industry. It is also designed to provide a safer environment for patients in the healthcare industry. Today, there are many life-threatening diseases that can be attained by a healthcare employee or a patient being exposed to a contaminated stethoscope head. There are incurable staphylococcus infections currently present in our environment today. By eliminating exposure to potentially contaminated stethoscope heads, the possibility of infection, and/or death due to exposure to a contaminated stethoscope head, is greatly reduced if not completely eliminated.

### BRIEF DESCRIPTION OF THE DRAWINGS/PHOTOGRAPHS

FIG. 1 is a large partial cross-sectional view of the cover in place on a bell portion of the head of a stethoscope.

FIG. 2 is a large partial cross-sectional view of the cover in place on a diaphragm portion of the head of a stethoscope.

FIG. 3 is a top view of a disposable cover for a stethoscope head for both the bell and diaphragm.

FIG. 4 is a top view of disposable cover for a stethoscope head for the diaphragm.

FIG. 5 is an aerial view of the disposable stethoscope cover actually in place on a bell.

FIG. 6 is an aerial view of the disposable stethoscope cover actually in place on a diaphragm.

FIG. 7 is a side view of the disposable stethoscope cover actually in place on both the bell and diaphragm.

FIG. 8 is an aerial view of the disposable stethoscope cover actually in place on both the bell and diaphragm.

### DETAILED DESCRIPTION OF INVENTION

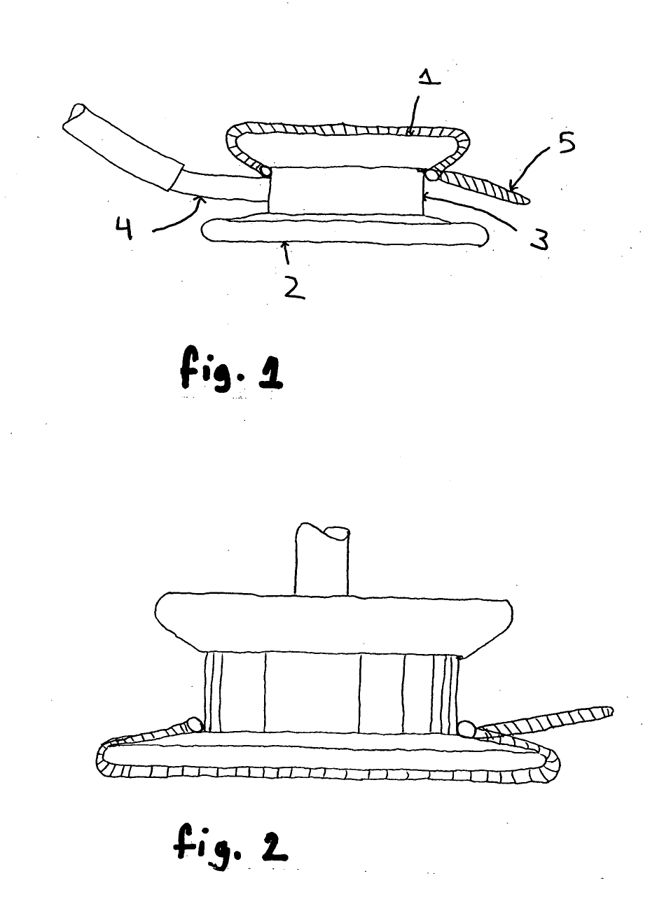

Referring to FIG. 2, the stethoscope head is comprised of the bell portion L, the diaphragm portion 2 and the neck portion 3 which separates the bell from the diaphragm and includes an opening for a tubal attachment 4 to the remaining parts of a stethoscope.

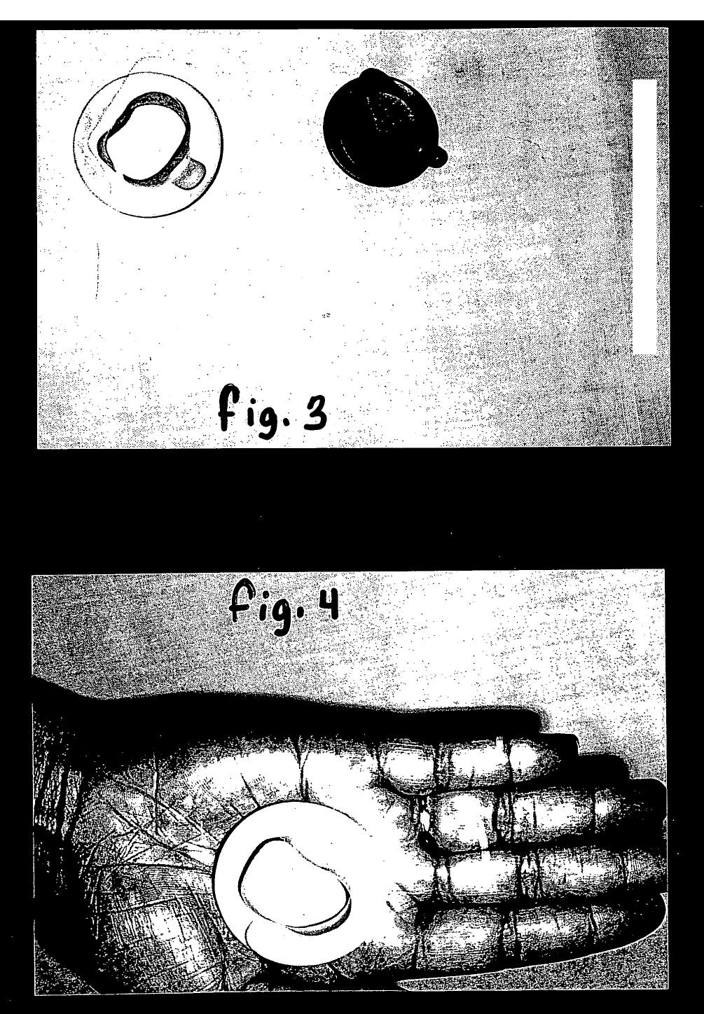

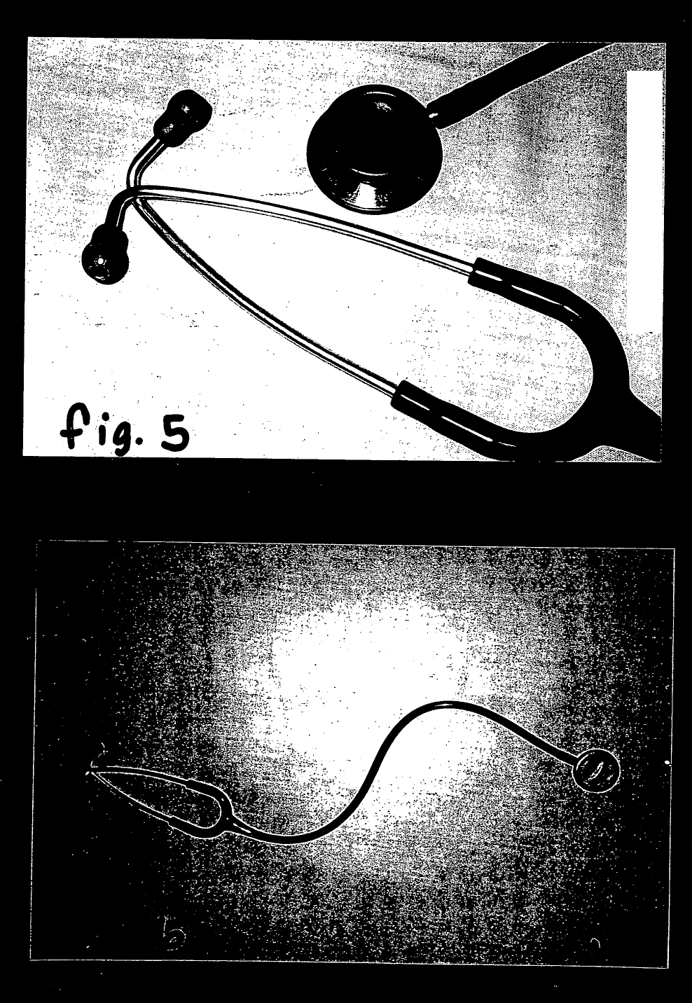

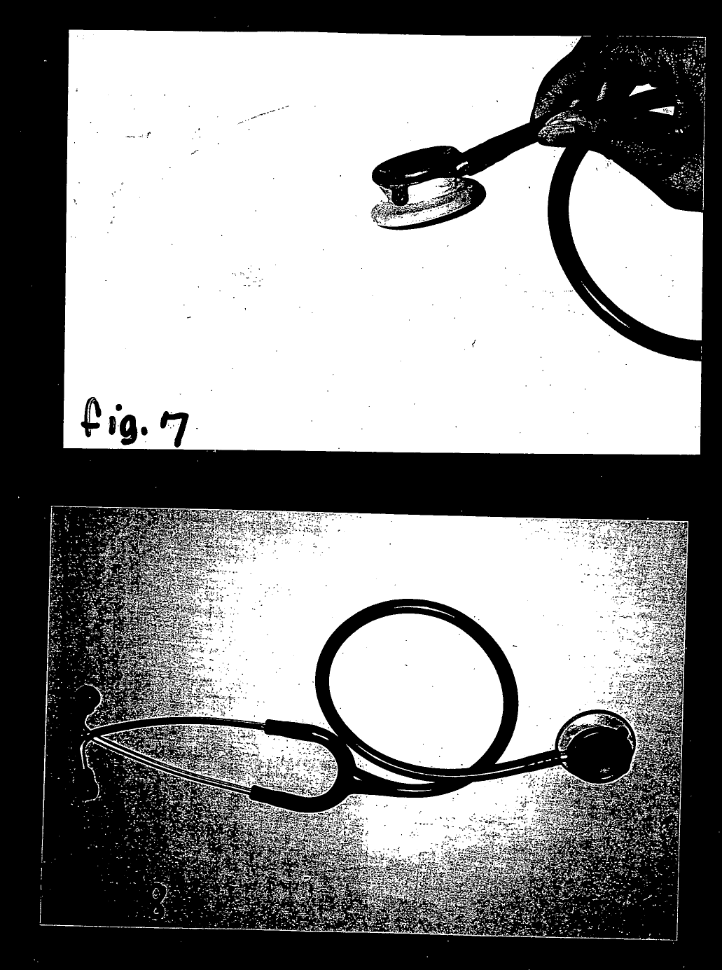

This invention is designed from a flexible, stretchable, non-permeable material that is designed to cover either the diaphragm or the bell of a stethoscope head prior to any contact with a patients skin. This cover is designed for a single use. In FIG. 3, the cover is designed to completely cover either the diaphragm or bell of a stethoscope head. The cover has an opening designed to stretch over the diaphragm or bell and then detract back to normal size to completely encase the diaphragm or bell when inserting and to do the same, once more, when removing. The cover has a tab 5, that could vary in size, shape, and material, made of a durable material designed to aide in insertion and removal of disposable cover. The tab will cause the cover to be inserted and removed with great ease. The tab can be easily seen and easily grasped during insertion and removal. While the exact measurements for the stethoscope head, and it's components, vary, this cover is designed to accommodate such variations.

There have been previous attempts to invent disposable covers for stethoscopes heads. In most previous cases, the covers were difficult to install and even more difficult to disengage after use. The present invention is designed to make it much easier to cover the head of a stethoscope with a disposable, non-permeable, cover that will prevent the spread of infections, diseases, body fluids, or organisms. The cover will be easy to install and ever more easier to remove. This will cause the medical profession to embrace this invention and use it with regularity with each patient. The Stethoscope head cover will include a highly visible, easily accessible tab that protrudes from the edge of the disposable stethoscope cover. This tab will enable the user to easily place the sterile/non-sterile, non-permeable, cover on the stethoscope head many times per day with great ease and to remove the cover, many times per day, with even greater ease.

The cover can be made with a form of plastic, rubber, paper, or other synthetic material with each material being elastic on the edges that will cover the stethoscope head allowing the cover to expand wide enough to fit over the head of the stethoscope and then to contract tightly for a snug fit on the neck of the stethoscope head. Each of these materials allow the cover to be sterile, or non-sterile, and non-permeable. Also, the material will also allow the user to maintain the highest quality of hearing frequency. The foregoing invention has been described with various embodiments thereof it will be appreciated that numerous variations, modifications, and embodiments are possible and accordingly, all such variations, modifications, and embodiments are regarded as being within the spirit and the scope of the invention as defined in the following claims:

1. A disposable stethoscope cover, with tab, made from paper, plastic, rubber, fabric, or any other elastomeric blend.

2. A disposable cover as claimed in claim 1, that can be made in one size to fit the bell and another size to fit the diaphragm.

3. A disposable stethoscope cover as claimed in claim 1, wherein the tab is made in any shape or size.

4. A disposable stethoscope cover as claimed in claim 1, wherein the tab can be made in the body of the cover or can be made as a detachable part of the cover that can be affixed after the cover is completed.

5. A disposable stethoscope cover as claimed in claim 1, wherein the cover can be made from various weights of elastomeric blend.
